(19) United States
(12) Patent Application Publication
(10) Pub. No.: US 2007/0292380 A1
(43) Pub. Date: Dec. 20, 2007

(54) Hair conditioning composition containing a non-guar galactomannan polymer derivative

(76) Inventors: James Anthony Staudigel, Loveland, OH (US); Marjorie Mossman Peffly, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/454,808

(22) Filed: Jun. 16, 2006

(51) Int. Cl. A61K 8/73; A61K 8/41; A61K 8/37; A61K 8/34

(52) U.S. Cl. 424/70.13; 424/70.12; 424/70.31; 424/70.27

(57) ABSTRACT

A hair conditioning composition comprising:
a) from about 0.01 wt. % to about 10 wt. % of a non-guar galactomannan polymer derivative having a mannose to galactose ratio of greater than 2:1 on a monomer to monomer basis, said non-guar galactomannan polymer derivative selected from the group consisting of a cationic non-guar galactomannan polymer derivative and an amphoteric non-guar galactomannan polymer derivative having a net positive charge;
   i. wherein said non-guar galactomannan polymer derivative has a molecular weight from about 1,000 to about 10,000,000; and
   ii. wherein said non-guar galactomannan polymer derivative has a cationic charge density from about 0.7 meq/g to about 7 meq/g;
b) a conditioning agent selected from the group consisting of cationic surfactants, cationic polymers, nonvolatile silicones, nonvolatile hydrocarbons, saturated C₁₄ to C₂₂ straight chain fatty alcohols, nonvolatile hydrocarbon esters, and mixtures thereof; and
c) wherein said hair conditioning composition is substantially free of an anionic surfactant.

### FIELD OF INVENTION

The present invention relates to hair conditioning compositions containing a non-guar galactomannan polymer derivative having a mannose to galactose ratio of greater than 2:1. More specifically, it relates to hair conditioning compositions containing a conditioning agent, a cationic or amphoteric non-guar galactomannan polymer derivative having a mannose to galactose ratio of greater than 2:1 and having a cationic charge density of from at least about 0.7 meq/g to about 7 meq/g.

### BACKGROUND OF THE INVENTION

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils and other natural conditioning and moisturizing components. The hair can further be left with increased levels of static upon drying, which can interfere with combing and result in a condition commonly referred to as “fly-away hair”, or contribute to an undesirable phenomena of “split ends”, particularly for long hair.

A variety of approaches have been developed to condition the hair after shampooing. A common method of providing conditioning benefit to the hair is through the use of hair conditioning agents such as cationic surfactants and polymers, high melting point fatty compounds, low melting point oils, silicone compounds, and mixtures thereof. Most of these conditioning agents are known to provide conditioning benefits by depositing on the hair. For example, silicone compounds are known to provide conditioning benefits such as softness and smoothness to the hair, by depositing on the hair.

It has been found that it is still not easy to obtain expected conditioning efficacy, especially conditioning benefits such as softness and smoothness and combability by application of traditional conditioning compositions.

Based on the foregoing, there remains a desire for hair conditioning compositions which provide improved conditioning agent deposition, thus providing improved conditioning benefits such as softness and smoothness and combability.

There also exists a desire for leave-in and rinse-off hair conditioning compositions which provide improved silicone deposition, while not deteriorating other benefits such as slippery feel and slick feel on wet hair.

### SUMMARY

The present invention is directed to a hair conditioning composition comprising:
a) from about 0.01 wt. % to about 10 wt. % of a non-guar galactomannan polymer derivative having a mannose to galactose ratio of greater than 2:1 on a monomer to monomer basis, said non-guar galactomannan polymer derivative selected from the group consisting of a cationic non-guar galactomannan polymer derivative and an amphoteric non-guar galactomannan polymer derivative having a net positive charge;
   i. wherein said non-guar galactomannan polymer derivative has a molecular weight from about 1,000 to about 10,000,000;
   ii. wherein said non-guar galactomannan polymer derivative has a cationic charge density from about 0.7 meq/g to about 7 meq/g;
b) a conditioning agent selected from the group consisting of cationic surfactants, cationic polymers, nonvolatile silicones, nonvolatile hydrocarbons, quartemary ammonium salts, saturated C₁₄ to C₂₂ straight chain fatty alcohols, nonvolatile hydrocarbon esters, and mixtures thereof; and
c) wherein said hair conditioning composition is substantially free of an anionic surfactant.

### DETAILED DESCRIPTION OF THE INVENTION

While the specification concludes with claims that particularly point out and distinctly claim the invention, it is believed the present invention will be better understood from the following description.

The hair conditioning compositions of the present invention comprise a cationic non-guar galactomannan polymer, a conditioning agent, and less than about 5% of an anionic surfactant. Each of these essential components, as well as preferred or optional components, is described in detail hereinafter.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

All percentages, parts, and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The term “charge density,” as used herein, refers to the ratio of the number of net positive charges on a monomeric unit of which a polymer is comprised to the molecular weight of said monomeric unit. The charge density multiplied by the polymer molecular weight determines the number of positively charged sites on a given polymer chain.

The term “substantially free of an anionic surfactant,” as used herein, means that the composition comprises less than 5% by weight of the composition of a detersive surfactant.

The term “polymer,” as used herein, shall include materials whether made by polymerization of one type of monomer or made by two (i.e., copolymers) or more types of monomers.

The term “water-soluble,” as used herein, means that the polymer is soluble in water in the present composition. In general, the polymer should be soluble at 25° C. at a concentration of at least 0.1% by weight of the water solvent, preferably at least 1%, more preferably at least 5%, most preferably at least 15%.

The term “water-insoluble,” as used herein, means that a compound is not soluble in water in the present composition. Thus, the compound is not miscible with water.

### Non-Guar Galactomannan Polymer Derivative

The hair conditioning compositions of the present invention comprise non-guar galactomannan polymer derivatives having a mannose to galactose ratio of greater than 2:1 on a monomer to monomer basis, the non-guar galactomannan polymer derivative selected from the group consisting of a cationic non-guar galactomannan polymer derivative and an amphoteric non-guar galactomannan polymer derivative having a net positive charge. As used herein, the term “cationic non-guar galactomannan” refers to a non-guar galactomannan polymer to which a cationic group is added. The term “amphoteric non-guar galactomannan” refers to a non-guar galactomannan polymer to which a cationic group and an anionic group are added such that the polymer has a net positive charge. It has been discovered that non-guar galactomannan polymer derivatives provide improved efficacy of conditioning agents. Enhanced conditioning benefits include increased silicone deposition, which results in improved hair smoothness and combability.

Non-guar galactomannan polymers are present in the endosperm of seeds of the Leguminosae family. Non-guar galactomannan polymers are made up of a combination of mannose monomers and galactose monomers. The non-guar galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and also is affected by climate. The non-guar galactomannan polymers of the present invention have a ratio of mannose to galactose of greater than 2:1 on a monomer to monomer basis (i.e., non-guar galactomannan polymers). Preferably, the ratio of mannose to galactose is greater than about 3:1, and more preferably the ratio of mannose to galactose is greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and cassia gum (5 parts mannose/1 part galactose).

The cationic non-guar galactomannan polymer derivatives for use in the hair conditioning compositions of the present invention have a molecular weight from about 1,000 to about 10,000,000. In one embodiment of the present invention, the cationic non-guar galactomannan polymer derivatives have a molecular weight from about 5,000 to about 3,000,000. As used herein, the term “molecular weight” refers to the weight average molecular weight. The weight average molecular weight may be measured by gel permeation chromatography.

The hair conditioning compositions of the present invention include non-guar galactomannan polymer derivatives which have a cationic charge density from about 0.7 meq/g to about 7 meq/g. In one embodiment of the present invention, the non-guar galactomannan polymer derivatives have a charge density from about 0.9 meq/g to about 7 meq/g. The degree of substitution of the cationic groups onto the non-guar galactomannan structure should be sufficient to provide the requisite cationic charge density.

In one embodiment of the present invention, the non-guar galactomannan polymer derivative is a cationic derivative of the non-guar galactomannan polymer, which is obtained by reaction between the hydroxyl groups of the non-guar galactomannan polymer and reactive quaternary ammonium compounds. Suitable quaternary ammonium compounds for use in forming the cationic non-guar galactomannan polymer derivatives include those conforming to the general formula:

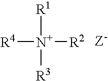

wherein R¹, R² and R³ are methyl or ethyl groups; R⁴ is either an epoxyalkyl group of the general formula:

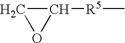

or R⁴ is a halohydrin group of the general formula:

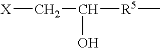

wherein R⁵ is a C₁ to C₃ alkylene; X is chlorine or bromine, and Z is an anion such as Cl⁻, Br⁻, I⁻ or HSO₄⁻.

Cationic non-guar galactomannan polymer derivatives formed from the reagents described above are represented by the general formula:

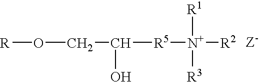

wherein R is the gum. Preferably, the cationic non-guar galactomannan derivative is a gum hydroxypropyltrimethylammonium chloride, which can be more specifically represented by the general formula:

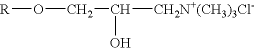

In another embodiment of the present invention, the non-guar galactomannan polymer derivative is an amphoteric non-guar galactomannan polymer derivative having a net positive charge, obtained when the cationic non-guar galactomannan polymer derivative further comprises an anionic group.

The hair conditioning compositions of the present invention comprise non-guar galactomannan polymer derivatives at a range of about 0.01% to about 10%, and more preferably from about 0.05% to about 5%, by weight of the composition.

### Conditioning Agents

The compositions of the present invention also comprise one or more conditioning agents, such as those selected from the group consisting of cationic surfactants, cationic polymers, nonvolatile silicones (including soluble and insoluble silicones), nonvolatile hydrocarbons, saturated C₁₄ to C₂₂ straight chain fatty alcohols, nonvolatile hydrocarbon esters, and mixtures thereof. Preferred conditioning agents are cationic surfactants, cationic polymers, saturated C₁₄ to C₂₂ straight chain fatty alcohols, quartemary ammonium salts and silicones (especially insoluble silicones). The components hereof can comprise from about 0.1% to about 99%, more preferably from about 0.5% to about 90%, of conditioning agents. However, in the presence of an aqueous carrier, the conditioning agents preferably comprise from about 0.1% to about 90%, more preferably from about 0.5 to about 60% and most preferably from about 1% to about 50% by weight of the hair conditioning composition.

### Cationic Surfactants

Cationic surfactants, useful in compositions of the present invention, contain amino or quaternary ammonium moieties. The cationic surfactant will preferably, though not necessarily, be insoluble in the compositions hereof. Cationic surfactants among those useful herein are disclosed in the following documents: M.C. Publishing Co., McCutcheon's, Detergents & Emulsifiers, (North American edition 1979); Schwartz, et al., Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin et al., issued Dec. 30, 1975; U.S. Pat. No. 3,959,461, Bailey et al., issued May 25, 1976; and U.S. Pat. No. 4,387,090, Bolich, Jr., issued Jun. 7, 1983.

Among the quaternary ammonium-containing cationic surfactant materials useful herein are those of the general formula:

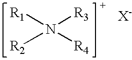

wherein R₁-R₄ are independently an aliphatic group of from about 1 to about 22 carbon atoms or an aromatic, alkoxy, polyoxyalkylene, alkylamido, hydroxyalkyl, aryl or alkylaryl group having from about 1 to about 22 carbon atoms; and X is a salt-forming anion such as those selected from halogen, (e.g., chloride, bromide), acetate, citrate, lactate, glycolate, phosphate nitrate, sulfate, and alkylsulfate radicals. The aliphatic groups may contain, in addition to carbon and hydrogen atoms, ether linkages, and other groups such as amino groups. The longer chain aliphatic groups, e.g., those of about 12 carbons, or higher, can be saturated or unsaturated. Especially preferred are di-long chain (e.g., di C₁₂-C₂₂, preferably C₁₆-C₁₈, aliphatic, preferably alkyl). di-short chain (e.g., C₁-C₃ alkyl, preferably C₁-C₂ alkyl) quaternary ammonium salts,

Salts of primary, secondary, and tertiary fatty amines are also suitable cationic surfactant materials. The alkyl groups of such amines preferably have from about 12 to about 22 carbon atoms, and may be substituted or unsubstituted. Such amines, useful herein, include stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (with 5 moles of ethylene oxide) stearylamine, dihydroxy ethyl stearylamine, and arachidylbehenylamine. Suitable amine salts include the halogen, acetate, phosphate, nitrate, citrate, lactate, and alkyl sulfate salts. Such salts include stearylamine hydrochloride, soyamine chloride, stearylamine formate, N-tallowpropane diamine dichloride and stearamidopropyl dimethylamine citrate. Cationic amine surfactants included among those useful in the present invention are disclosed in U.S. Pat. No. 4,275,055, Nachtigal, et al., issued Jun. 23, 1981.

Cationic surfactants are preferably utilized at levels of from about 0.1% to about 10%, more preferably from about 0.25% to about 5%, most preferably from about 0.5% to about 2%, by weight of the composition.

### Cationic Polymer Conditioning Agent

In addition to the non-guar galactomannan derivatives, the compositions of the present invention can also comprise one or more additional cationic polymer conditioning agents. The cationic polymer conditioning agents will preferably be water soluble. Cationic polymers are typically used in the same ranges as disclosed above for cationic surfactants.

The cationic polymers hereof will generally have a weight average molecular weight which is at least about 5,000, typically at least about 10,000, and is less than about 10 million. Preferably, the molecular weight is from about 100,000 to about 2 million. The cationic polymers will generally have cationic nitrogen-containing moieties such as quaternary ammonium or cationic amino moieties, and mixtures thereof.

The cationic charge density is preferably at least about 0.1 meq/g, more preferably at least about 1.5 meq/g, even more preferably at least abut 1.1 meq/g, most preferably at least about 1.2 meq/g. The “cationic charge density” of a polymer, as that term is used herein, refers to the ratio of the number of positive charges on a monomeric unit of which the polymer is comprised to the molecular weight of said monomeric unit. The cationic charge density multiplied by the polymer molecular weight determines the number of positively charged sites on a given polymer chain. The average molecular weight of such suitable cationic polymers will generally be between about 10,000 and 10 million, preferably between about 50,000 and about 5 million, more preferably between about 100,000 and about 3 million. Those skilled in the art will recognize that the charge density of amino-containing polymers may vary depending upon pH and the isoelectric point of the amino groups. The charge density should be within the above limits at the pH of intended use.

Any anionic counterions can be utilized for the cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

The cationic nitrogen-containing moiety will be present generally as a substituent, on a fraction of the total monomer units of the cationic hair conditioning polymers. Thus, the cationic polymer can comprise copolymers, terpolymers, etc. of quaternary ammonium or cationic amine-substituted monomer units and other non-cationic units referred to herein as spacer monomer units. Such polymers are known in the art, and a variety can be found in the CTFA Cosmetic Ingredient Dictionary, 3rd edition, edited by Estrin, Crosley, and Haynes, (The Cosmetic, Toiletry, and Fragrance Association, Inc., Washington, D.C., 1982).

Suitable cationic polymers include, for example, copolymers of vinyl monomers having cationic amine or quaternary ammonium functionalities with water soluble spacer monomers such as acryl-amide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Other suitable spacer monomers include vinyl esters, vinyl alcohol (made by hydrolysis of poly-vinyl acetate), maleic anhydride, propylene glycol, and ethylene glycol.

The cationic amines can be primary, secondary, or tertiary amines, depending upon the particular species and the pH of the composition. In general, secondary and tertiary amines, especially tertiary amines, are preferred.

Amine-substituted vinyl monomers can be polymerized in the amine form, and then optionally can be converted to ammonium by a quaternization reaction. Amines can also be similarly quaternized subsequent to formation of the polymer. For example, tertiary amine functionalities can be quaternized by reaction with a salt of the formula R′X wherein R′ is a short chain alkyl, preferably a C₁-C₇ alkyl, more preferably a C₁-C₃ alkyl, and X is an anion which forms a water soluble salt with the quaternized ammonium.

Suitable cationic amino and quaternary ammonium monomers include, for example, vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts. The alkyl portions of these monomers are preferably lower alkyls such as the C₁-C₃ alkyls, more preferably C₁ and C₂ alkyls. Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

The cationic polymers hereof can comprise mixtures of monomer units derived from amine- and/or quaternary ammonium-substituted monomer and/or compatible spacer monomers.

Suitable cationic hair conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and copolymers of unsaturated carboxylic acids having from 3 to 5 carbon atoms, as described in U.S. Pat. No. 4,009,256.

Other cationic polymers that can be used include polysaccharide polymers, such as cationic cellulose derivatives and cationic starch derivatives.

Cationic polysaccharide polymer materials suitable for use herein include those of the formula:

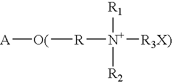

wherein: A is an anhydroglucose residual group, such as a starch or cellulose anhydroglucose residual, R is an alkylene oxyalkylene, polyoxyalkylene, or hydroxyalkylene group, or combination thereof, R₁, R₂, and R₃ independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R₁, R₂ and R₃) preferably being about 20 or less, and X is an anionic counterion, as previously described.

Cationic cellulose is available from Amerchol Corp. (Edison, N.J., USA) in their Polymer JR® and LR® series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted opoxide, referred to in the industry (CTFA) as Polyquaternium 24. These materials are available from Amerchol Corp. (Edison, N.J., USA) under the tradename Polymer LM-200®.

Other cationic polymers that can be used include cationic guar gum derivatives, such as guar hydroxypropyltrimonium chloride (commercially available from Celanese Corp. in their Jaguar R series). Other materials include quaternary nitrogen-containing cellulose ethers (e.g., as described in U.S. Pat. No. 3,962,418), and copolymers of etherified cellulose and starch (e.g., as described in U.S. Pat. No. 3,958,581).

As discussed above, the cationic polymer hereof is water soluble. This does not mean, however, that it must be soluble in the composition. Preferably however, the cationic polymer is either soluble in the composition, or in a complex coacervate phase in the composition formed by the cationic polymer and anionic material. Complex coacervates of the cationic polymer can be formed with anionic surfactants or with anionic polymers that can optionally be added to the compositions hereof (e.g., sodium polystyrene sulfonate). However, the present hair conditioning composition is substantially free of anionic surfactants. Where anionic surfactants are present, they are used only in amounts of less than about 5%, preferably less than about 3% and most preferably less than about 2% by weight of the hair conditioning composition.

### Silicone Conditioning Agents

The compositions hereof can also include nonvolatile soluble or insoluble silicone conditioning agents. By soluble what is meant is that the silicone conditioning agent is miscible with the aqueous carrier of the composition so as to form part of the same phase. By insoluble what is meant is that the silicone forms a separate, discontinuous phase from the aqueous carrier, such as in the form of an emulsion or a suspension of droplets of the silicone.

The silicone hair conditioning agent will be used in the compositions hereof at levels of from about 0.05% to about 20% by weight of the composition, preferably from about 0.1% to about 10%, more preferably from about 0.5% to about 5%, most preferably from about 0.5% to about 3%.

Soluble silicones include silicone copolyols, such as dimethicone copolyols, e.g., polyether siloxane-modified polymers, such as polypropylene oxide, polyethylene oxide modified polydimethylsiloxane, wherein the level of ethylene and/or propylene oxide sufficient to allow solubility in the composition.

Preferred, however, are insoluble silicones. The insoluble silicone hair conditioning agent for use herein will preferably have viscosity of from about 1,000 to about 2,000,000 centistokes at 25° C., more preferably from about 10,000 to about 1,800,000, even more preferably from about 100,000 to about 1,500,000. The viscosity can be measured by means of a glass capillary viscometer as set forth in Dow Corning Corporate Test Method CTM0004, Jul. 20, 1970.

Suitable insoluble, nonvolatile silicone fluids include polyalkyl siloxanes, polyaryl siloxanes, polyalkylaryl siloxanes, polyether siloxane copolymers, and mixtures thereof. Other insoluble, nonvolatile silicone fluids having hair conditioning properties can also be used. The term “nonvolatile” as used herein shall mean that the silicone has a boiling point of at least about 260° C., preferably at least about 275° C., more preferably at least about 300° C. Such materials exhibit very low or no significant vapor pressure at ambient conditions. The term “silicone fluid” shall mean flowable silicone materials having a viscosity of less than 1,000,000 centistokes at 25° C. Generally, the viscosity of the fluid will be between about 5 and 1,000,000 centistokes at 25° C., preferably between about 10 and about 300,000 centistokes.

Silicone fluids hereof also include polyalkyl or polyaryl siloxanes with the following structure:

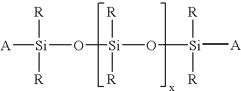

wherein R is alkyl or aryl, and x is an integer from about 7 to about 8,000 may be used. “A” represents groups which block the ends of the silicone chains.

The alkyl or aryl groups substituted on the siloxane chain (R) or at the ends of the siloxane chains (A) may have any structure as long as the resulting silicones remain fluid at room temperature, are hydrophobic, are neither irritating, toxic nor otherwise harmful when applied to the hair, are compatible with the other components of the composition, are chemically stable under normal use and storage conditions, and are capable of being deposited on and of conditioning hair.

Suitable A groups include methyl, methoxy, ethoxy, propoxy, and aryloxy. The two R groups on the silicone atom may represent the same group or different groups. Preferably, the two R groups represent the same group. Suitable R groups include methyl, ethyl, propyl, phenyl, methylphenyl and phenylmethyl. The preferred silicones are polydimethyl siloxane, polydiethylsiloxane, and polymethylphenylsiloxane. Polydimethylsiloxane is especially preferred.

The nonvolatile polyalkylsiloxane fluids that may be used include, for example, polydimethylsiloxanes. These siloxanes are available, for example, from the General Electric Company in their ViscasilR and SF 96 series, and from Dow Corning in their Dow Corning 200 series.

The polyalkylaryl siloxane fluids that may be used, also include, for example, polymethylphenylsiloxanes. These siloxanes are available, for example, from the General Electric Company as SF 1075 methyl phenyl fluid or from Dow Corning as 556 Cosmetic Grade Fluid.

Especially preferred, for enhancing the shine characteristics of hair, are highly arylated silicones, such as highly phenylated polyethyl silicone having refractive indices of about 1.46 or higher, especially about 1.52 or higher. When these high refractive index silicones are used, they should be mixed with a spreading agent, such as a surfactant or a silicone resin, as described below to decrease the surface tension and enhance the film forming ability of the material.

The polyether siloxane copolymers that may be used include, for example, a polypropylene oxide modified polydimethylsiloxane (e.g., Dow Corning DC-1248) although ethylene oxide or mixtures of ethylene oxide and propylene oxide may also be used. The ethylene oxide and polypropylene oxide level should be sufficiently low to prevent solubility in the composition hereof.

References disclosing suitable silicone fluids include U.S. Pat. No. 2,826,551, Geen; U.S. Pat. No. 3,964,500, Drakoff, issued Jun. 22, 1976; U.S. Pat. No. 4,364,837, Pader; and British Patent No. 849,433, Woolston. Also useful are Silicon Compounds distributed by Petrarch Systems, Inc., 1984. This reference provides an extensive (though not exclusive) listing of suitable silicone fluids.

Another silicone hair conditioning material that can be especially useful in the silicone conditioning agents is insoluble silicone gum. The term “silicone gum”, as used herein, means polyorganosiloxane materials having a viscosity at 25° C. of greater than or equal to 1,000,000 centistokes. Silicone gums are described by Petrarch and others including U.S. Pat. No. 4,152,416, Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. The “silicone gums” will typically have a mass molecular weight in excess of about 200,000, generally between about 200,000 and about 1,000,000. Specific examples include polydimethylsiloxane, (polydimethylsiloxane) (methylvinylsiloxane) copolymer, poly(dimethylsiloxane) (diphenyl siloxane)(methylvinylsiloxane) copolymer and mixtures thereof.

Preferably the silicone hair conditioning agent comprises a mixture of a polydimethylsiloxane gum, having a viscosity greater than about 1,000,000 centistokes and polydimethylsiloxane fluid having a viscosity of from about 10 centistokes to about 100,000 centistokes, wherein the ratio of gum to fluid is from about 30:70 to about 70:30, preferably from about 40:60 to about 60:40.

An optional ingredient that can be included in the silicone conditioning agent is silicone resin. Silicone resins are highly crosslinked polymeric siloxane systems. The crosslinking is introduced through the incorporation of trifunctional and tetrafunctional silanes with monofunctional or difunctional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units (and hence, a sufficient level of crosslinking) such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and tetra-chlorosilane, with the methyl-substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art.

Silicone resins can enhance deposition of silicone on the hair and can enhance the glossiness of hair with high refractive index volumes.

Background material on silicones including sections discussing silicone fluids, gums, and resins, as well as manufacture of silicones, can be found in Encyclopedia of Polymer Science and Engineering, Volume 15, Second Edition, pp 204-308, John Wiley & Sons, Inc., 1989.

Silicone materials and silicone resins in particular, can conveniently be identified according to a shorthand nomenclature system well known to those skilled in the art as “MDTQ” nomenclature. Under this system, the silicone is described according to presence of various siloxane monomer units which make up the silicone. Briefly, the symbol M denotes the monofunctional unit (CH₃)₃SiO)_{0.5}; D denotes the difunctional unit (CH₃)₂SiO; T denotes the trifunctional unit (CH₃)SiO_{1.5}; and Q denotes the quadric- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyls, amines, hydroxyls, etc. The molar ratios of the various units, either in terms of subscripts to the symbols indicating the total number of each type of unit in the silicone (or an average thereof) or as specifically indicated ratios in combination with molecular weight complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

### Aqueous Carrier

The compositions of the present invention may also comprise an aqueous carrier. Preferably, the aqueous carrier is present in an amount of from about 50% to about 99.8% by weight of the hair conditioning composition. The aqueous carrier comprises a water phase which can optionally include other liquid, water-miscible or water-soluble solvents such as lower alkyl alcohols, e.g. C₁-C₅ alkyl monohydric alcohols, preferably C₂-C₃ alkyl alcohols. However, the liquid fatty alcohol must be miscible in the aqueous phase of the composition. Said fatty alcohol can be naturally miscible in the aqueous phase or can be made miscible through the use of cosolvents or surfactants.

In one embodiment, the composition is an emulsion, having viscosity at 25° C. of at least about 5,000 cP preferably from about 8,000 cP to about 50,000 cP, more preferably from about 15,000 cP to about 35,000 cP. Viscosity is determined by a Brookfield RVT, at 20 RPM.

### Anti-Dandruff Actives

The hair conditioning compositions may also comprise an anti-dandruff active. Suitable non-limiting examples of anti-dandruff actives include pyridinethione salts (i.e., zinc pyrithione), azoles, selenium sulfide, particulate sulfur, keratolytic agents, and mixtures thereof. Such anti-dandruff actives should be physically and chemically compatible with the essential components of the composition, and should not otherwise unduly impair product stability, aesthetics or performance.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Azole anti-microbials include imidazoles such as climbazole and ketoconazole.

Selenium sulfide compounds are described, for example, in U.S. Pat. No. 2,694,668; U.S. Pat. No. 3,152,046; U.S. Pat. No. 4,089,945; and U.S. Pat. No. 4,885,107.

Sulfur may also be used as a particulate anti-microbial/anti-dandruff agent in the hair conditioning compositions.

The hair conditioning compositions may further comprise one or more keratolytic agents such as salicylic acid.

Additional anti-microbial actives may include extracts of melaleuca (tea tree) and charcoal.

### Particles

The hair conditioning compositions may also comprise particles. Useful particles can be natural, inorganic, synthetic, or semi-synthetic. In the present invention, it is preferable to incorporate no more than about 20%, more preferably no more than about 10% and even more preferably no more than 2%, by weight of the composition, of particles. In one embodiment, the particles have an average mean particle size of less than about 300 μm.

Non-limiting examples of natural particles comprise hydrophobic tapioca starch, corn starch, and dried fruit particles.

Non-limiting examples of inorganic particles include colloidal silicas, fumed silicas, precipitated silicas, silica gels, magnesium silicate, glass particles, talcs, micas, sericites, and various natural and synthetic clays including bentonites, hectorites, and montmorillonites.

Examples of synthetic particles comprise silicone resins, poly(meth)acrylates, polyethylene, polyester, polypropylene, polystyrene, polyurethane, polyamide (e.g., Nylon®), epoxy resins, urea resins, acrylic powders, and the like.

Non-limiting examples of hybrid particles include sericite & crosslinked polystyrene hybrid powder, and mica and silica hybrid powder.

### Other Ingredients

The compositions herein can contain a variety of other optional components suitable for rendering such compositions more cosmetically or aesthetically acceptable or to provide them with additional usage benefits. Such conventional optional ingredients are well-known to those skilled in the art.

A wide variety of additional ingredients can be formulated into the present composition. These include: other conditioning agents; hair-hold polymers; detersive surfactants such as anionic, nonionic, amphoteric, and zwitterionic surfactants; additional thickening agents and suspending agents such as xanthan gum, guar gum, hydroxyethyl cellulose, methyl cellulose, hydroxyethylcellulose, starch and starch derivatives; viscosity modifiers such as methanolamides of long chain fatty acids such as cocomonoethanol amide; crystalline suspending agents; pearlescent aids such as ethylene glycol distearate; preservatives such as benzyl alcohol, methyl paraben, propyl paraben and imidazolidinyl urea; polyvinyl alcohol; ethyl alcohol; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents, such as the thioglycolates; perfumes; sequestering agents, such as disodium ethylenediamine tetra-acetate; and polymer plasticizing agents, such as glycerin, disobutyl adipate, butyl stearate, and propylene glycol. Such optional ingredients generally are used individually at levels from about 0.01% to about 10.0%, preferably from about 0.05% to about 5.0% by weight of the composition.

### Method of Use

The hair conditioning compositions of the present invention are used in conventional ways to provide the conditioning and other benefits of the present invention. Such method of use depends upon the type of composition employed but generally involves application of an effective amount of the product to the hair, which may then be rinsed from the hair (as in the case of hair rinses) or allowed to remain on the hair (as in the case of gels, lotions, and creams). “Effective amount” means an amount sufficient enough to provide a dry combing benefit. In general, from about 1 g to about 50 g is applied to the hair on the scalp. The composition is distributed throughout the hair, typically by rubbing or massaging the hair and scalp. Preferably, the composition is applied to wet or damp hair prior to drying of the hair. After such compositions are applied to the hair, the hair is dried and styled in accordance with the preference of the user. In the alternative, the composition is applied to dry hair, and the hair is then combed or styled in accordance with the preference of the user.

### EXAMPLES

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention.

All parts, percentages, and ratios herein are by weight unless otherwise specified. Some components may come from suppliers as dilute solutions. The levels given reflect the weight percent of the active material, unless otherwise specified.

| Material INCI Name | Quantity |
|---|---|
| Alcohol denat. | 10.00 |
| Cassia hydroxypropyltrimonium chloride¹ | 0.05 |
| Panthenol | 0.05 |
| Ethylhexyl Methoxycinnamate | 0.10 |
| Fragrance | 0.30 |
| Dimethicone (and) Dimethiconol² | 40.00 |
| Dimethicone³ | 5.00 |
| DIMETHICONE⁴ | 10.00 |
| Isododecane⁵ | 34.50 |

¹Cationic Cassia EX906, Noveon Inc. (Brecksville, OH, USA) MW 300,000 and 4.25% N.
²Dow Corning 1403, Dow Corning Corp. (Midland, MI, USA).
³Silkonoel AK 500, Wacker Silicones Corp. (Adrian, Mich. USA)
⁴Dow Corning 1403, Dow Corning Corp. (Midland, MI, USA).
⁵Permethyl 99, Presperse Inc. (Somerset, NJ, USA)

| Material INCI Name | Quantity |
|---|---|
| Water/Aqua | 94.8770 |
| Cassia hydroxypropyltrimonium chloride¹ | 0.05 |
| Amodimethicone and Cetrimonium | |
| Chloride and Trideceth-12² | 1.928 |
| Polyquaternium-11³ | 1.335 |
| PEG-40 Hydrogenated Castor Oil | 0.500 |
| PPG-2 Methyl Ether | 0.500 |
| DMDM Hydantoin | 0.370 |
| Disodium EDTA and Water | 0.140 |
| Polysorbate 80 | 0.120 |
| Aminomethyl Propanol | 0.100 |
| Citric Acid | 0.080 |

¹Cationic Cassia EX906, Noveon Inc. (Brecksville, OH, USA)
²Dow Corning 949 Cationic Emulsion, Dow Corning Corp. (Midland, MI, USA).
³Gaffquat 755N-H, ISP Corp. (Wayne, NJ, USA)

| Material INCI Name | Quantity |
|---|---|
| Water | 94.75 |
| Cassia Hydroxypropyltrimonium Chloride¹ | 0.05 |
| Guar Hydroxypropyltrimonium Chloride² | 0.50 |
| Cetearyl alcohol, PEG 20 Stearate³ | 0.50 |
| Cetyl Alcohol | 0.10 |
| Sodium Benzoate | 0.50 |
| Glyoxylic Acid⁴ | 0.25 |
| Creatine⁵ | 0.10 |
| Behentrimonium Chloride⁶ | 0.80 |
| Polyquaternium-32 and Mineral Oil and | 2.00 |
| PPG-1 Trideceth-6⁷ | |
| Panthenol | 0.05 |
| Hydrolyzed Silk⁸ | 0.10 |
| Fragrance | 0.30 |

¹Cationic Cassia EX906, Noveon Inc. (Brecksville, OH, USA)
²Jaguar C-13, Rhodia, Inc. (Cranbury, NJ, USA)
³Polawax GP200, Croda, Inc. (Edison, NJ, USA)
⁴GA 50%, DSM Fine Chemicals (Linz, Austria)
⁵Tego Cosmo C100, Degussa Goldschmidt (Hopewell, NJ, USA)
⁶Genamin KDMP, Clariant GmbH (Sulzbach, Germany)
⁷Salcare SC92, Ciba Specialty Chemicals, Inc. (Highpoint, NC, USA)
⁸Silkpro Liquid, Ikeda Corporation, (Japan)

| Material INCI Name | Quantity |
|---|---|
| Water/Aqua | q.s. |
| Cassia hydroxypropyltrimonium chloride¹ | 0.05 |
| Stearyl Alcohol² | 4.643 |
| Dimethicone³ | 4.200 |
| Behentrimonium chloride/ | 2.848 |
| IsopropylAlcohol⁴ | |
| Cetyl Alcohol⁵ | 1.857 |
| Benzyl Alcohol | 0.400 |
| Fragrance/Parfume | 0.350 |
| Disodium Ethylene Diamine Tetraacetic | 0.127 |
| Acid | |
| Panthenyl Etheyl Ether | 0.030 |
| Panthenol | 0.0534 |
| Sodium Hydroxide | 0.0136 |
| Methylchloroisothiazolinone, | 0.033 |
| Methylisothiazolinone | |

¹Cationic Cassia EX906, Noveon Inc. (Brecksville, OH, USA)
²CO-1895, Procter & Gamble Company. (Cincinnati, OH, USA)
³GE Silicones (Waterford, NY, USA)
⁴Genamin KDMP, Clariant GmbH (Sulzbach, Germany)
⁵CO-1695, Procter & Gamble Company. (Cincinnati, OH, USA)

| Material INCI Name | Quantity |
|---|---|
| Water/Aqua | q.s. |
| Cassia hydroxypropyltrimonium chloride¹ | 0.05 |
| Glutamic Acid² | 0.640 |
| Behenamidopropyl Dimethylamine³ | 2.300 |
| Cetyl Alcohol⁴ | 2.500 |
| Stearyl Alcohol⁵ | 4.500 |
| Dimethicone, Cyclopentasiloxane⁶ | 4.200 |
| EDTA | 0.100 |
| Benzyl Alcohol | 0.400 |
| Methylchloroisothiazolinone, | 0.033 |
| Methylisothiazolinone | |
| Fragrance/Parfume | 0.350 |
| Panthenyl Etheyl Ether | 0.050 |
| Panthenol | 0.089 |

¹Cationic Cassia EX906, Noveon Inc. (Brecksville, OH, USA)
²L-Glutamic Acid, Orsan/Amylum (Nesle, France)
³Incrimine BB, Croda Inc. (Edison, NJ, USA)
⁴CO-1695, Procter & Gamble Company. (Cincinnati, OH, USA)
⁵CO-1895, Procter & Gamble Company. (Cincinnati, OH, USA)
⁶CF1213 Fluid, GE Silicones (Waterford, NY, USA)

| Material INCI Name | Quantity |
|---|---|
| Water/Aqua | q.s. |
| Cassia hydroxypropyltrimonium chloride¹ | 0.05 |
| Citric Acid | 0.120 |
| Stearamidopropyl Dimethylamine² | 1.000 |
| Quaternium-18³ | 0.750 |
| Hydroxyethylcellulose (HEC)⁴ | 0.250 |
| Cetyl Alcohol⁵ | 0.200 |
| Stearyl Alcohol⁶ | 0.750 |
| Cetearyl Alcohol/Polysorbate 60⁷ | 0.500 |
| Glyceryl Stearate⁸ | 0.250 |
| Oleyl Alcohol⁹ | 0.250 |
| Amodimeticone¹⁰ | 0.500 |
| EDTA | 0.100 |
| Benzyl Alcohol | 0.400 |
| Methylchloroisothiazolinone, | 0.033 |
| Methylisothiazolinone | |
| Fragrance/Parfume | 0.400 |
| Panthenyl Etheyl Ether | 0.050 |
| Panthenol | 0.089 |

¹Cationic Cassia EX906, Noveon Inc. (Brecksville, OH, USA)
²SAPDMA, Inolex Chemical Company (Philadelphia, PA, USA)
³Varisoft 442 100 P, Goldschmidt Degussa (Hopewell, VA, USA)
⁴Cellosize, Dow Chemical (Piscataway, NJ USA)
⁵CO-1695, Procter & Gamble Company. (Cincinnati, OH, USA)
⁶CO-1895, Procter & Gamble Company. (Cincinnati, OH, USA)
⁷Polawax NF, Croda Inc. (Edison, NJ, USA)
⁸Stepan GMS Pure, Stepan (Northfield, IL, USA)
⁹Novol, Croda Inc. (Edison, NJ USA)
¹⁰GE Silicones (Waterford, NY, USA)

| Material INCI Name | Quantity | | |
|---|---|---|---|
| Water/Aqua | q.s. | q.s. | q.s. |
| Cassia hydroxypropyltrimonium chloride¹ | 0.05 | 0.05 | 0.05 |
| Stearyl Alcohol² | 4.643 | 4.643 | 4.643 |
| Dimethicone³ | 4.200 | 4.200 | 4.200 |
| Behentrimonium chloride/Isopropyl | 2.848 | 2.848 | 2.848 |
| Alcohol⁴ | | | |
| Cetyl Alcohol⁵ | 1.857 | 1.857 | 1.857 |
| ZPT⁶ | 1.000 | — | — |
| Climbazole⁷ | — | 1.000 | — |
| Salicylic Acid⁸ | — | — | 1.000 |
| Benzyl Alcohol | 0.400 | 0.400 | 0.400 |
| Fragrance/Parfume | 0.350 | 0.350 | 0.350 |
| Disodium Ethylene Diamine Tetraacetic | 0.127 | 0.127 | 0.127 |
| Acid | | | |
| Panthenyl Etheyl Ether | 0.030 | 0.030 | 0.030 |
| Panthenol | 0.0534 | 0.0534 | 0.0534 |
| Sodium hydroxide | 0.0136 | 0.0136 | 0.0136 |
| Methylchloroisothiazolinone, | 0.033 | 0.033 | 0.033 |
| Methylisothiazolinone | | | |

¹Cationic Cassia EX906, Noveon Inc. (Brecksville, OH, USA)
²CO-1895, Procter & Gamble Company. (Cincinnati, OH, USA)
³GE Silicones (Waterford, NY, USA)
⁴Genamin KDMP, Clariant GmbH (Sulzbach, Germany)
⁵CO-1695, Procter & Gamble Company. (Cincinnati, OH, USA)
⁶U2 Zinc Pyrithone, Arch Chemicals Inc. (Norwalk, CT, USA)
⁷Crinipan USP, Symrise GmbH & Co. (Germany)
⁸Salicylic Acid, Sigma-Aldrich (St. Louis, MO, USA)

| Material INCI Name | Quantity | | |
|---|---|---|---|
| Water/Aqua | q.s. | q.s. | q.s. |
| Cassia hydroxypropyltrimonium chloride¹ | 0.05 | 0.05 | 0.05 |
| Glutamic Acid² | 0.640 | 0.640 | 0.640 |
| Behenamidopropyl Dimethylamine³ | 2.300 | 2.300 | 2.300 |
| Cetyl Alcohol⁴ | 2.500 | 2.500 | 2.500 |
| Stearyl Alcohol⁵ | 4.500 | 4.500 | 4.500 |
| Dimethicone, Cyclopentasiloxane⁶ | 4.200 | 4.200 | 4.200 |
| ZPT⁷ | 1.000 | — | — |
| Climbazole⁸ | — | 1.000 | — |
| Salicylic Acid⁹ | — | — | 1.000 |
| EDTA | 0.100 | 0.100 | 0.100 |
| Benzyl Alcohol | 0.400 | 0.400 | 0.400 |
| Methylchloroisothiazolinone, | 0.033 | 0.033 | 0.033 |
| Methylisothiazolinone | | | |
| Fragrance/Parfume | 0.350 | 0.350 | 0.350 |
| Panthenyl Etheyl Ether | 0.050 | 0.050 | 0.050 |
| Panthenol | 0.089 | 0.089 | 0.089 |

¹Cationic Cassia EX906, Noveon Inc. (Brecksville, OH, USA)
²L-Glutamic Acid, Orsan/Amylum (Nesle, France)
³Incrimine BB, Croda Inc. (Edison, NJ, USA)
⁴CO-1695, Procter & Gamble Company. (Cincinnati, OH, USA)
⁵CO-1895, Procter & Gamble Company. (Cincinnati, OH, USA)
⁶CF1213 Fluid, GE Silicones (Waterford, NY, USA)
⁷U2 Zinc Pyrithone, Arch Chemicals Inc. (Norwalk, CT, USA)
⁸Crinipan USP, Symrise GmbH & Co. (Germany)
⁹Salicylic Acid, Sigma-Aldrich (St. Louis, MO, USA)

| Material INCI Name | Quantity | | |
|---|---|---|---|
| Water/Aqua | q.s. | q.s. | q.s. |
| Cassia hydroxypropyltrimonium chloride¹ | 0.05 | 0.05 | 0.05 |
| Citric Acid | 0.120 | 0.120 | 0.120 |
| Stearamidopropyl Dimethylamine² | 1.000 | 1.000 | 1.000 |
| Quaternium-18³ | 0.750 | 0.750 | 0.750 |
| Hydroxyethylcellulose (HEC)⁴ | 0.250 | 0.250 | 0.250 |
| Cetyl Alcohol⁵ | 0.200 | 0.200 | 0.200 |
| Stearyl Alcohol⁶ | 0.750 | 0.750 | 0.750 |
| Cetearyl Alcohol/Polysorbate 60⁷ | 0.500 | 0.500 | 0.500 |
| Glyceryl Stearate⁸ | 0.250 | 0.250 | 0.250 |
| Oleyl Alcohol⁹ | 0.250 | 0.250 | 0.250 |
| Amodimeticone¹⁰ | 0.500 | 0.500 | 0.500 |
| ZPT¹¹ | 1.000 | — | — |
| Climbazole¹² | — | 1.000 | — |
| Salicylic Acid¹³ | — | — | 1.000 |
| EDTA | 0.100 | 0.100 | 0.100 |
| Benzyl Alcohol | 0.400 | 0.400 | 0.400 |
| Methylchloroisothiazolinone, | 0.033 | 0.033 | 0.033 |
| Methylisothiazolinone | | | |
| Fragrance/Parfume | 0.400 | 0.400 | 0.400 |
| Panthenyl Etheyl Ether | 0.050 | 0.050 | 0.050 |
| Panthenol | 0.089 | 0.089 | 0.089 |

¹Cationic Cassia EX906, Noveon Inc. (Brecksville, OH, USA)
²SAPDMA, Inolex Chemical Company (Philadelphia, PA, USA)
³Varisoft 442 100 P, Goldschmidt Degussa (Hopewell, VA, USA)
⁴Cellosize, Dow Chemical (Piscataway, NJ USA)
⁵CO-1695, Procter & Gamble Company. (Cincinnati, OH, USA)
⁶CO-1895, Procter & Gamble Company. (Cincinnati, OH, USA)
⁷Polawax NF, Croda Inc. (Edison, NJ, USA)
⁸Stepan GMS Pure, Stepan (Northfield, IL, USA)
⁹Novol, Croda Inc. (Edison, NJ USA)
¹⁰GE Silicones (Waterford, NY, USA)
¹¹U2 Zinc Pyrithone, Arch Chemicals Inc. (Norwalk, CT, USA)
¹²Crinipan USP, Symrise GmbH & Co. (Germany)
¹³Salicylic Acid, Sigma-Aldrich (St. Louis, MO, USA)

It is understood that the examples and embodiments described herein are for illustrative purposes only and that various modifications or changes in light thereof will be suggested to one skilled in the art without departing from the scope of the present invention.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioning composition comprising:
a) from about 0.01 wt. % to about 10 wt. % of a non-guar galactomannan polymer derivative having a mannose to galactose ratio of greater than 2:1 on a monomer to monomer basis, said non-guar galactomannan polymer derivative selected from the group consisting of a cationic non-guar galactomannan polymer derivative and an amphoteric non-guar galactomannan polymer derivative having a net positive charge;
i. wherein said non-guar galactomannan polymer derivative has a molecular weight from about 1,000 to about 10,000,000; and
ii. wherein said non-guar galactomannan polymer derivative has a cationic charge density from about 0.7 meq/g to about 7 meq/g;
b) a conditioning agent selected from the group consisting of cationic surfactants, cationic polymers, nonvolatile silicones, nonvolatile hydrocarbons, quartemary ammonium salts, saturated C₁₄ to C₂₂ straight chain fatty alcohols, nonvolatile hydrocarbon esters, and mixtures thereof; and
c) wherein said hair conditioning composition is substantially free of an anionic surfactant.

2. A hair conditioning composition according to claim 1, wherein said non-guar galactomannan polymer derivative has a cationic charge density from about 0.9 meq/g to about 7 meq/g.

3. A hair conditioning composition according to claim 1, wherein said non-guar galactomannan polymer derivative has a molecular weight from about 5,000 to about 3,000,000.

4. A hair conditioning composition according to claim 1, wherein said conditioning agent is a fatty alcohol.

5. A hair conditioning composition according to claim 1, wherein said non-volatile silicone is an insoluble silicone.

6. A hair conditioning composition according to claim 1, wherein said non-guar galactomannan polymer derivative is cationic cassia.

7. A hair conditioning composition according to claim 1, further comprising an anti-dandruff active.

8. A hair conditioning composition according to claim 7, wherein said anti-dandruff active is selected from the group consisting of zinc pyrithione, climbazole, and salicylic acid.

9. A hair conditioning composition according to claim 1, further comprising an aqueous carrier.

10. A hair conditioning composition according to claim 1, further comprising particles.

11. A hair conditioning composition according to claim 9, wherein said particles are natural particles.
